(19) United States
(12) Patent Application Publication
(10) Pub. No.: US 2026/0108642 A1
(43) Pub. Date: Apr. 23, 2026

(54) USE OF EXOSOME, COMPLEX COMPRISING EXOSOME, TRACER, AND PREPARATION METHOD THEREOF

(71) Applicant: National Atomic Research Insitute, Taoyuan (TW)

(72) Inventors: MEI-HUI WANG, Taoyuan (TW); Kai-Hung Cheng, Taoyuan (TW); Chen-Hsin Chan, Taoyuan (TW); Chung-Li Ho, Taoyuan (TW); Hung-Wen Yu, Taoyuan (TW); Yi-Shan Chen, Taoyuan (TW)

(21) Appl. No.: 18/969,548

(22) Filed: Dec. 5, 2024

(30) Foreign Application Priority Data
Oct. 18, 2024 (TW) 113139754

(51) Int. Cl. A61K 51/12; A61K 51/04; A61P 25/28; A61P 35/04; B82Y 5/00

(52) U.S. Cl. CPC A61K 51/1203; A61K 51/0482; A61P 25/28; A61P 35/04; A61K 2121/00; A61K 2123/00; B82Y 5/00

(57) ABSTRACT

Provided herein are use of an exosome, a complex comprising an exosome, a tracer, and a preparation method and use thereof. The complex comprises an exosome, a bifunctional chelating agent coupled to the exosome, and a radionuclide gallium-67 or gallium-68 embedded on the bifunctional chelating agent. In a preferred implementation, the bifunctional chelating agent comprises 1,4,7,10-tetraazacyclododecane-1,4,7,10-tetraacetic acid (DOTA).

### CROSS REFERENCE TO RELATED APPLICATION

This application claims the benefits of Taiwan application Serial No. 113139754, filed on Oct. 18, 2024, the disclosures of which are incorporated by references herein in its entirety.

### BACKGROUND

### Technical Field

The present invention relates to the field of radiological imaging and diagnosis, and in particular, to an exosome labeled with a radionuclide.

### Related Art

Autologous mesenchymal stem cell therapy has shown significant results in bone marrow transplantation, neurodegenerative diseases, and cancers, but allogeneic mesenchymal stem cell therapy may show immunotoxicity. Exosomes are a type of extracellular vesicles. There is growing evidence that mesenchymal stem cell-derived exosomes carry signaling factors such as nucleic acids, proteins, carbohydrates, and lipids of mesenchymal stem cells, but they are less likely to cause rejection, which are safer. In addition, exosomes are natural transporters and have the advantages of high biocompatibility, ability to penetrate deep tissues and pass through biological barriers, and ability to transport various substances. This makes it possible for exosome therapy to replace cell therapy.

Internationally, there are two main methods for labeling exosomes. One method is labeling proteins on the surface of exosomes. An exosomal surface protein label comprises an iodine label or an indium-111 label. To know the distribution of iodine-labeled exosomes in an organism, it is usually necessary to sacrifice the animal, take out organs, and read the activity accumulated in each organ, and indium-111 labeling is inefficient, so both of them have their limitations in application. The other labeling method is carrying a radioactive metal (such as indium-111 or zirconium-89) by an ion carrier (for example, 8-hydroxyquinoline or oxine) into exosomes, but the amount of radioactivity into the exosomes by this method is not high.

In view of this, there is an urgent need in the art to provide a novel exosome labeled with a radioactive substance or a labeling method, to overcome the defect in the related art.

### SUMMARY

For readers to understand the basic meaning of the present disclosure, the summary provides a brief description of the present disclosure. The summary is not a complete description of the present disclosure and is not intended to define the technical features or scope of the claims of the present invention.

According to a first implementation of the present invention, a complex comprises an exosome, a bifunctional chelating agent, and a radionuclide. Specifically, the bifunctional chelating agent is coupled to the exosome, where the bifunctional chelating agent comprises 1,4,7,10-tetraazacyclododecane-1,4,7,10-tetraacetic acid (DOTA), and the radionuclide is gallium-67 or gallium-68.

In an optional implementation, the bifunctional chelating agent is p-NCS-Bn-DOTA-GA or p-NCS-Bn-DOTA. In a specific implementation, the bifunctional chelating agent is p-NCS-Bn-DOTA-GA.

According to a second implementation of the present invention, an exosome tracer comprises the complex according to any one of the foregoing implementations and a pharmaceutically acceptable carrier.

In addition, according to a third implementation of the present invention, a method for preparing the complex according to any one of the foregoing implementations comprises the following steps:
(a) mixing 50-150 nm of exosome with a bifunctional chelating agent for at least three hours to obtain a mixture;
(b) filtering out the excess bifunctional chelating agent to obtain an exosome binding to a bifunctional chelating group; and
(c) adding the exosome binding to the bifunctional chelating group and a radionuclide into an acidic buffer solution, prior to shaking for at least 10 minutes, to obtain the complex, wherein the radionuclide is gallium-67 or gallium-68. In an optional implementation, a pH value of the acidic buffer solution is about 4-5.

According to an implementation of the present invention, the 50-150 nm of exosome is isolated from cells, for example, isolated from mesenchymal stem cells. Further, a method for preparing the 50-150 nm of exosome comprises the following steps:
(a-1) collecting, by a tangential flow filtration system, a medium with the mesenchymal stem cells cultured, and collecting the 50-150 nm of exosome with a flat sheet membrane and a hollow fiber membrane; and
(a-2) purifying the 50-150 nm of exosome by column chromatography.

In a preferred implementation, in step (a) in the method of the present invention, the 50-150 nm of exosome and the bifunctional chelating agent are mixed for at least five hours, and further, in the step of radionuclide labeling, the exosome binding to the bifunctional chelating group and the radionuclide are shaken for at least 15 minutes to obtain the complex.

In addition, according to a fourth implementation of the present invention, provided is use of the complex according to any one of the foregoing implementations in preparing a pharmaceutical composition targeting a lesion for treating Alzheimer's disease or leukemia with an exosome or imaging osteoporosis with exosome labeled with gallium radioisotope.

A person of ordinary skill in the technical field to which the present invention belongs can fully understand the central concept, adopted technical means, and various implementations of the present invention with reference to the following implementations.

### BRIEF DESCRIPTION OF THE DRAWINGS

To make the foregoing descriptions of the present invention and other objectives, features, advantages, and embodiments more apparent and understandable, the drawings are described as follows:

FIG. 1 shows the results of labeling efficiency of a complex of the present invention gallium-67 exosome and exosomes labeled with other radionuclides according to an example of the present invention;

FIG. 2 shows the results of temporal stability of a complex of the present invention gallium-67 exosome according to an example of the present invention;

FIG. 3 shows the distribution of a complex of the present invention gallium-67 exosome (the exosome derived from umbilical cord mesenchymal stem cells) as a tracer in a wild-type mouse (wild type) and a mouse with Alzheimer's disease (AD mice) according to another example of the present invention;

FIG. 4 shows the distribution of a complex of the present invention gallium-67 exosome (the exosome derived from bone marrow mesenchymal stem cells) as a tracer in a wild-type mouse (wild type) and a mouse with Alzheimer's disease (Alzheimer's group) according to still another example of the present invention;

FIG. 5 shows the distribution of a complex of the present invention gallium-67 exosome (the exosome derived from umbilical cord mesenchymal stem cells) as a tracer in a wild-type mouse (WT) and a mouse with cerebellar atrophy (SCA) according to another example of the present invention;

FIG. 6 shows the change of amyloid deposition in the hippocampus of the brain of a mouse with Alzheimer's disease after four weeks of treatment with an exosome derived from umbilical cord mesenchymal stem cells according to an example of the present invention;

FIG. 7 shows that the addition of an exosome derived from umbilical cord mesenchymal stem cells to Reh leukemia cells effectively inhibits the growth of 70% of Reh leukemia cells according to an example of the present invention; and

FIG. 8 shows that the intravenous injection of an exosome derived from umbilical cord mesenchymal stem cells into a mouse with Reh leukemia cells effectively prolongs the life span of the mouse with the Reh leukemia cells by one week according to an example of the present invention.

### DETAILED DESCRIPTION

To make the description of the present disclosure more exhaustive and complete, the following provides illustrative textual descriptions for the implementations and specific embodiments of the present invention, but the implementations and specific embodiments of the present invention are not limited thereto.

Unless otherwise indicated, scientific and technical terms used in this specification have the same meaning as understood and commonly used by a person of ordinary skill in the art. Further, the noun used in this specification cover both the singular and plural forms of the noun, unless otherwise indicated.

As described in this specification, the word “about” usually means that an actual value is plus or minus 10%, 5%, 1%, or 0.5% of a specific value or range. The word “about” herein means that an actual value falls within an acceptable standard error of a mean, depending on the consideration of a person of ordinary skill in the technical field to which the present invention belongs. With the exception of examples, or unless otherwise expressly stated, it is to be understood that the ranges, quantities, values, and percentages herein are modified by the word “about”. Accordingly, unless otherwise indicated, the values or parameters disclosed in this specification and in the appended claims are all approximate values and may be changed as required.

The word “individual” refers to an animal comprising a human, suitable for the exosome, complex, and tracer of the present invention. Unless specifically noted, the word “individual” is intended to indicate both males and females.

In some specific implementations, the complex of the present invention may be administered in combination with a pharmaceutically acceptable carrier. The “pharmaceutically acceptable carrier” herein means a pharmaceutically acceptable carrier, diluent, or excipient compatible with the complex of the present invention and harmless to the individual. A person of ordinary skill in the technical field to which the present invention belongs may make adjustments according to a mode of administration and a standard pharmaceutical specification based on actual use. Further, the complex of the present invention may be prepared in a variety of different dosage forms according to technical standards for pharmaceutical formulations, comprising but not limited to injections, tablets, suppositories, drops, or suspensions.

The complex of the present invention can be administered to an individual through various routes of administration, comprising but not limited to: oral administration, parenteral administration, sublingual administration, transdermal administration, enteral administration, transmucosal administration, topical administration, inhalation administration, buccal administration, intrapleural administration, intravenous administration, intra-arterial administration, peritoneal administration, subcutaneous administration, intramuscular administration, intranasal administration, intraspinal administration, intra-articular administration, or a combination thereof.

For parenteral administration, the complex of the present invention may be used with an appropriate pharmaceutically acceptable carrier or diluent, comprising but not limited to water, oils, ethanol, saline, phosphate buffer, glycerol, or glycol (such as propylene glycol or polyethylene glycol). In addition, a stabilizer, an antioxidant, and/or a preservative may also be added.

In the present disclosure, the word “treating or treatment” comprises partially or completely preventing, ameliorating, mitigating, and/or handling symptoms or signs associated with Alzheimer's disease or leukemia. The treatment is “effective” when one or more symptoms or clinical markers are reduced. Alternatively, the treatment is “effective” when a course of a symptom, secondary disease or sign is slowed or discontinued.

A dose of the complex of the present invention administered to an individual may be determined based on actual use, as long as a therapeutic effect on the individual can be achieved. A specific dose is adjusted based on an active ingredient, a dosage form, an age and weight of a patient, a mode of administration, and time of administration. In an implementation, the complex is provided in unit doses, and each unit dose comprises an effective amount of active ingredient. In a non-limiting implementation, for the dose of the complex of the present invention administered to the individual, a person of ordinary skill in the technical field to which the present invention belongs should understand that a human equivalent dose (HED) of a drug may be calculated based on a dose of in an animal mode.

To resolve the existing problem in the related art, the present invention provides a novel exosome, complex, and tracer for use in the field of radiological diagnosis and/or treatment, and a method for preparing the complex.

The complex of the present invention comprises an exosome, a bifunctional chelating agent containing DOTA, and a radionuclide gallium-68 or gallium-67. The exosome is coupled to the bifunctional chelating agent and labeled with the radionuclide. In a specific embodiment, the bifunctional chelating agent is p-NCS-Bn-DOTA-GA.

According to another implementation of the present invention, the complex and a pharmaceutically acceptable carrier are mixed to prepare an exosome tracer.

In addition, according to still another implementation of the present invention, a method for preparing the complex is provided. The surface of the exosome is modified with the bifunctional chelating agent by a chemical synthesis method, and labeled with gallium-68 or gallium-67 with the labeling efficiency of the radionuclide by the preparation method of the present invention up to 97%. In this case, the complex may be used as an exosome tracer for SPECT/PET, and the purified complex still has 99% or more of radiochemical purity when stored at room temperature or at 4° C. In the present invention, the method for preparing the complex comprises the following steps:
(a) mixing 50-150 nm of exosome with a bifunctional chelating agent for at least three hours to obtain a mixture;
(b) filtering out the excess bifunctional chelating agent to obtain an exosome binding to a bifunctional chelating group; and
(c) adding the exosome binding to the bifunctional chelating group and a radionuclide into a slightly acidic buffer solution, prior to shaking for at least 10 minutes, to obtain the complex, wherein the radionuclide is gallium-67 or gallium-68.

According to an implementation of the present invention, the 50-150 nm of exosome is isolated from mesenchymal stem cells. Further, the preparation method comprises the following steps:
(a-1) collecting, by a tangential flow filtration system, a medium with the mesenchymal stem cells cultured, and collecting the 50-150 nm of exosome with a flat sheet membrane and a hollow fiber membrane; and
(a-2) purifying the 50-150 nm of exosome by column chromatography.

In an optional implementation, the bifunctional chelating agent is p-NCS-Bn-DOTA-GA or p-NCS-Bn-DOTA, preferably p-NCS-Bn-DOTA-GA.

Further, in an implementation, in step (a) in the preparation method of the present invention, the 50-150 nm of exosome and the bifunctional chelating agent are mixed for at least five hours, such as 5, 6, 7, 8, 9, or 10 hours, and the exosome binding to the bifunctional chelating group and the radionuclide are shaken for at least 10 minutes, such as 10, 15, 20, 25, or 30 minutes, to obtain the complex of the present invention. According to a specific implementation of the present invention, if the exosome and the radionuclide are shaken for 15 minutes, the labeling efficiency may be up to 97%.

In addition, the pH value of the slightly acidic buffer solution used in step (c) in this preparation method is preferably 4.0-5.5, more preferably 4.0-5.0. In a specific implementation, the slightly acidic buffer solution is a sodium acetate buffer.

It needs to be noted that the exosome in the complex of the present invention is derived from cells, such as mesenchymal stem cells or cancer cells. A person of ordinary skill in the technical field to which the present invention belongs may isolate the exosome from a specific cell source according to the preparation method of the present invention as required.

Another advantage of the present invention is that the complex of the present invention is suitable for use as an exosome tracer. The complex can pass through the blood-brain barrier and accumulate in cerebellar atrophy lesions and in the hippocampus in Alzheimer's disease. Further, the complex can also be used as an exosome tracer for leukemia cells to track the location of the leukemia cells and assess the distribution of leukemia in the body. In addition, the exosome also accumulates in the osteoporotic regions in mice with Alzheimer's disease. Based on this, the complex of the present invention has the potential to be used as a tracer for assessing disease severity.

For another advantage of the present invention, it was unexpectedly found in the present invention that the complex of the present invention has the ability of targeting Alzheimer's disease and leukemia lesions and that the co-morbidity of Alzheimer's disease and osteoporosis exists, so that the complex is suitable for use in pharmaceuticals that also have the potential to target lesions and treat specific diseases. Results of examples show that a complex prepared with an exosome derived from umbilical cord mesenchymal stem cells has therapeutic efficacy for Alzheimer's disease and leukemia.

A plurality of examples is disclosed as follows to describe various different implementations of the present invention, so that a person of ordinary skill in the technical field to which the present invention belongs can implement the technical content disclosed in the present invention according to the disclosure of this specification. Therefore, the examples disclosed as follows may not be used to limit the scope of the claims of the present invention. Further, all references in this specification are deemed to be fully incorporated by reference into this specification.

### EXAMPLE 1 PREPARATION OF THE EXOSOME OF THE PRESENT INVENTION

The exosome in this example was isolated from a medium with umbilical cord mesenchymal stem cells cultured. The umbilical cord mesenchymal stem cells were first cultured in MSC NutriStem® XF Medium, the medium with the cells cultured was collected, and floating cells and cell debris were removed by using a 0.22 μm filter. For the collection of the exosome, 50-150 nm of exosome was collected directly from the cell culture medium by using a tangential flow filtration (TFF) system with a flat sheet membrane Minimate TFF Capsule with Omega 100K membrane and hollow fiber membranes HBM-TFF-EVs and HBM-TFF-MV, and then purified by using a mixed-mode column (anion exchange and molecular sieve) HiTrap Capto Core 700. Then, the exosome was purified by molecular sieve chromatography filled with Sepharose CL-6B.

### EXAMPLE 2 PREPARATION OF THE COMPLEX OF THE PRESENT INVENTION-GALLIUM-67 EXOSOME

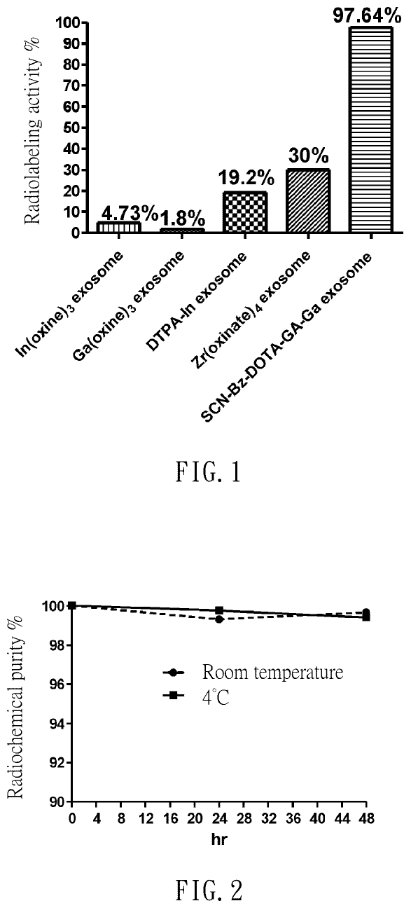

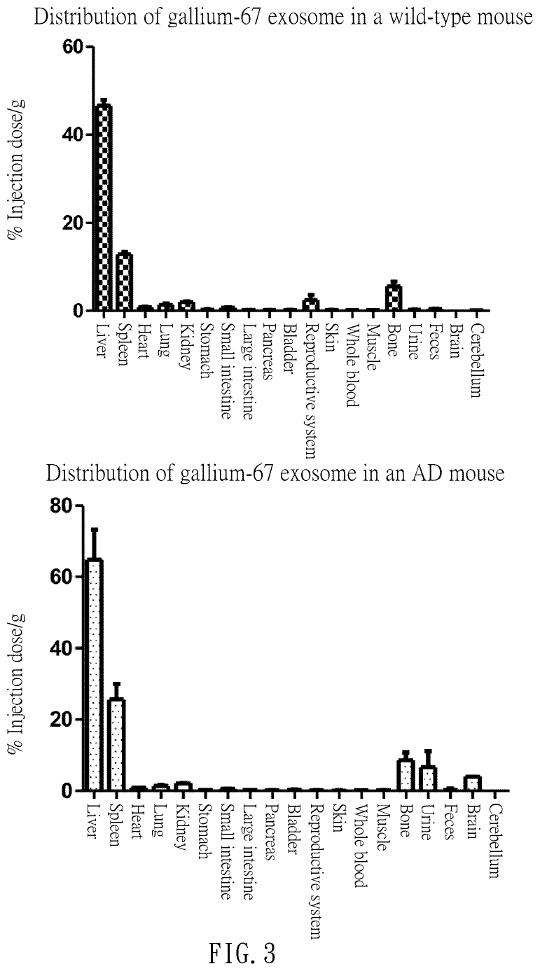

The collected exosome (about 2×10¹⁰ particles) was concentrated through a 100 kDa filtering membrane to 20 μL, dissolved in 0.2 mL of 0.1 N NaHCO₃ (pH 8.5), subjected to a bonding reaction with 10 mg of bifunctional chelating agent p-NCS-Bn-DOTA-GA for 5 h, then subjected to molecular sieve chromatography filled with Sepharose CL-6B or with a 100 kDa filtering membrane to remove excess p-NCS-Bn-DOTA-GA, and then concentrated through a 100 kDa filtering membrane to 20 μL. The exosome modified with a bifunctional chelating group was mixed with gallium-67 or gallium-68 in a 0.1 M sodium acetate buffer (pH 5) and shaken at room temperature for 15 min, to obtain the complex of the present invention (that is, SCN-Bz-DOTA-GA-Ga exosome in the figure). The measured radiolabeling activity is about 97.64%, and the complex can be stored for 48 h or more. The results are shown in FIG. 1 and FIG. 2.

### COMPARATIVE EXAMPLE 1 BONDING OF THE EXOSOME TO P-NCS-BN-DTPA AND INDIUM-111 RADIOLABELING

The collected exosome (about 2×10¹⁰ particles) was concentrated through a 100 kDa filtering membrane to 20 μL, dissolved in 0.2 mL of 0.1 N NaHCO₃ (pH 8.5), subjected to a bonding reaction with 10 mg of bifunctional chelating agent p-NCS-Bn-DTPA for 5 h, then subjected to molecular sieve chromatography with a 100 kDa filtering membrane to remove excess p-NCS-Bn-DTPA, and then concentrated through a 100 kDa filtering membrane to 20 μL. The exosome modified with a chelating group was mixed with indium-111 in 0.1 M citric acid (pH 2.0) and shaken at room temperature for 15 min, to obtain an exosome labeled with indium-111. The measured radiolabeling activity is about 19.2%.

### COMPARATIVE EXAMPLE 2 EXOSOME AND GALLIUM-67 OXINE RADIOLABELING

20 mg/mL (ethanol) of oxine was prepared. 20 μL of oxine was added to 166 μL of 1 M NaOAc and 0.025 mL of gallium-67 (2 N HCl; 12.21 mCi), and shaken at 50° C. to react for 30 min. After the reaction was completed, 200 μL of chloroform was added for extraction. After the extraction was completed, the lower solution (9.06 mCi) was taken and analyzed by TLC (iTLC; 100% EtOH or chloroform: MeOH=95:5). The solution was blown dry with argon, and 10 μL of EtOH was added for redissolving. The redissolved ⁶⁷Ga-Oxine (4.2 mCi) was added into the exosome and shaken at room temperature for 10 min, and then shaken ultrasonically for 3-10 s (this step was repeated once), to obtain a sample. The sample was subjected to ultra-high-speed centrifugation at 150000 g for 1 h. The measured radiolabeling activity is about 1.8%.

### COMPARATIVE EXAMPLE 3 INDIUM-111 OXINE-EXOSOME RADIOLABELING

The oxine was dissolved in 200 mM HEPES-buffered saline (HBS) at pH 7-7.5 to form a 1 mg/mL solution. 70-100 MBq indium-111 was added to 2 μg (2 μL) of oxine to form an indium-111 oxine complex. The indium-111 oxine complex was added into the exosome (1×10¹¹ particles) diluted in PBS to a final oxine concentration of 5 μg/mL, and incubated at 37° C. for 20 min. The measured radiolabeling activity is about 4.73%.

### COMPARATIVE EXAMPLE 4 ZR-89 OXINE

Refer to the information published by Rafael T. M. de Rosales (2022) in Bioconjug Chem 33(3): 473-485. The brief description is as follows: 70-100 MBq Zr-89 chloride was obtained from an accelerator and blown dry with nitrogen, and dissolved with 40 μg (0.3 μmol) of oxine (in 1 M HEPES) and 1 mg/mL polysorbate-80 at pH 7.8 into a 1 mg/mL solution, to form a Zr-89(oxinate)₄ complex. The Zr-89 oxinate complex was added into the exosome diluted in PBS, and incubated at 37° C. for 20 min. The measured radiolabeling activity is about 30%.

The tests in Comparative Examples 1-4 indicate that the exosome modified with p-NCS-Bn-DTPA or the exosome radiolabeled with indium-111 oxine or Zr-89 oxine has limited radiolabeling efficiency, lower than that of the complex of the present invention. By contrast, the results of Example 2 confirm that the radiolabeling technique of labeling gallium-67 into the exosome in the present invention provides radiolabeling efficiency up to 97% and only 15 minutes of radiolabeling reaction, without purification, which is advantageous to industrial application. Moreover, the complex of the present invention has radiation conditions suitable for SPECT (in the case of gallium-67 labeling) or PET (in the case of gallium-68 labeling) imaging, and thus has the potential to be used as an exosome tracer for SPECT/PET.

### EXAMPLE 3 DISTRIBUTION OF THE COMPLEX OF THE PRESENT INVENTION GALLIUM-67 P-NCS-BN-DOTA-GA-EXOSOME IN THE BRAIN OF A MOUSE WITH ALZHEIMER'S DISEASE

30 μCi gallium-67 p-NCS-Bn-DOTA-GA-exosome obtained in Example 2 was intravenously injected into the mouse with Alzheimer's disease, and the distribution of the exosome tracer derived from umbilical cord mesenchymal stem cells in the mouse with Alzheimer's disease was observed. It was found that the exosome tracer derived from the umbilical cord mesenchymal stem cells can pass through the blood-brain barrier and accumulate in the brain region of the mouse with Alzheimer's disease. The results are shown in FIG. 3.

### EXAMPLE 4 DISTRIBUTION OF THE COMPLEX OF THE PRESENT INVENTION GALLIUM-67 P-NCS-BN-DOTA-GA-EXOSOME IN THE REGION OF LEUKEMIA

A Reh leukemia exosome was isolated from a medium with Reh leukemia cells cultured. The Reh cells were cultured in a 10% FBS RPMI medium until 80% confluency and centrifuged at 2000 rpm for 5 min. After the supernatant was removed, the Reh cells were transferred to a serum free RPMI medium to be cultured for 24 h. The medium with the cells cultured was collected, and floating cells and cell debris were removed by using a 0.22 μm filter. For the collection of the exosome, 50-150 nm of exosome was collected directly from the cell culture medium by using a tangential flow filtration (TFF) system with a flat sheet membrane Minimate TFF Capsule with Omega 100K membrane and hollow fiber membranes HBM-TFF-EVs and HBM-TFF-MV, and then purified by using a mixed-mode column (anion exchange and molecular sieve) HiTrap Capto Core 700. Then, the exosome was purified by molecular sieve chromatography filled with Sepharose CL-6B. The collected exosome (about 2×10¹⁰ particles) was concentrated through a 100 kDa filtering membrane to 20 μL, dissolved in 0.2 mL of 0.1 N NaHCO₃ (pH 8.5), subjected to a bonding reaction with 10 mg of bifunctional chelating agent p-NCS-Bn-DOTA-GA for 5 h, then subjected to molecular sieve chromatography filled with Sepharose CL-6B with a 100 kDa filtering membrane to remove excess p-NCS-Bn-DOTA-GA, and then concentrated through a 100 kDa filtering membrane to 20 μL. The exosome modified with a bifunctional chelating group was mixed with gallium-67 or gallium-68 in a 0.1 M sodium acetate buffer (pH 5) and shaken at room temperature for 15 min, to obtain the complex of the present invention. The measured radiolabeling activity is about 97%. Through the comparison between the results of injecting the complex (that is, radiolabeled gallium-67 p-NCS-Bn-DOTA-GA) of the present invention as the exosome tracer intravenously into a mouse with leukemia and the distribution of F-18 FDG injected intravenously into the body of a mouse with leukemia, it was found that F-18 FDG was mainly distributed in the limbs of the mouse with leukemia, and the complex of the present invention also reached the limbs of the mouse with leukemia. As F-18 FDG mainly traces the location of cancer, it can be proved that the complex of the present invention can be used as a tracer for leukemia.

### EXAMPLE 5 DISTRIBUTION OF GALLIUM-67 P-NCS-BN-DOTA-GA-EXOSOME DERIVED FROM BONE MARROW MESENCHYMAL STEM CELLS IN THE PRESENT INVENTION IN THE REGION OF OSTEOPOROSIS

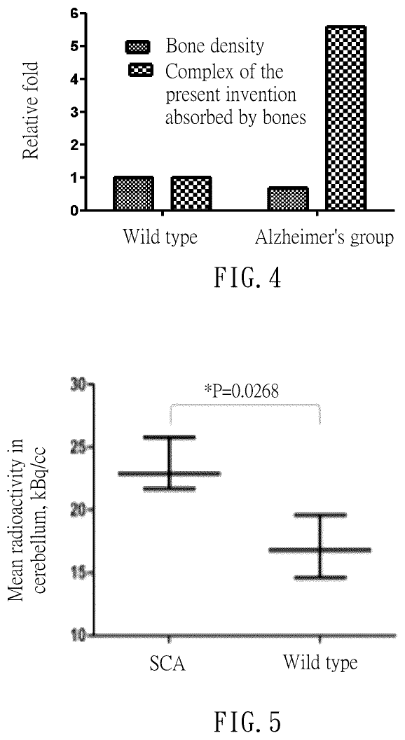

An exosome derived from bone marrow mesenchymal stem cells was isolated from a medium with the bone marrow mesenchymal stem cells cultured. The bone marrow mesenchymal stem cells were cultured in αMEM+16.6% FBS (834 mL αMEM+166 mL FBS) until 80% confluency and centrifuged at 2000 rpm for 5 min. After the supernatant was removed, the bone marrow mesenchymal stem cells were transferred to a serum free RPMI medium to be cultured for 24 h. The medium with the cells cultured was collected, and floating cells and cell debris were removed by using a 0.22 μm filter. For the collection of the exosome, 50-150 nm of exosome was collected directly from the cell culture medium by using a tangential flow filtration (TFF) system with a flat sheet membrane Minimate TFF Capsule with Omega 100K membrane and hollow fiber membranes HBM-TFF-EVs and HBM-TFF-MV, and then purified by using a mixed-mode column (anion exchange and molecular sieve) HiTrap Capto Core 700. Then, the exosome was purified by molecular sieve chromatography filled with Sepharose CL-6B. The collected exosome (about 2×10¹⁰ particles) was concentrated through a 100 kDa filtering membrane to 20 μL, dissolved in 0.2 mL of 0.1 N NaHCO₃ (pH 8.5), subjected to a bonding reaction with 10 mg of bifunctional chelating agent p-NCS-Bn-DOTA-GA for 5 h, then subjected to molecular sieve chromatography filled with Sepharose CL-6B with a 100 kDa filtering membrane to remove excess p-NCS-Bn-DOTA-GA, and then concentrated through a 100 kDa filtering membrane to 20 μL. The exosome modified with a bifunctional chelating group was mixed with gallium-67 or gallium-68 in a 0.1 M sodium acetate buffer (pH 5) and shaken at room temperature for 15 min, to obtain the complex of the present invention. The measured radiolabeling activity is about 97%. The radiolabeled gallium-67 p-NCS-Bn-DOTA-GA-exosome was injected intravenously into a wild-type mouse and a mouse with Alzheimer's disease. It was known that the mouse with Alzheimer's disease has poor bone density. The results show that the gallium-67 p-NCS-Bn-DOTA-GA-bone marrow mesenchymal stem cell-derived exosome highly accumulates at the location of poor bone density in the mouse with Alzheimer's disease. The results are shown in FIG. 4. The results show that the gallium-67 p-NCS-Bn-DOTA-GA-bone marrow mesenchymal stem cell-derived exosome can be used as a tracer for osteoporosis.

### EXAMPLE 6 DISTRIBUTION OF GALLIUM-67 P-NCS-BN-DOTA-GA-EXOSOME DERIVED FROM UMBILICAL CORD MESENCHYMAL STEM CELLS IN THE PRESENT INVENTION IN THE REGION OF CEREBELLAR ATROPHY

The exosome derived from the umbilical cord mesenchymal stem cells was isolated from a medium with the umbilical cord mesenchymal stem cells cultured. The umbilical cord mesenchymal stem cells were first cultured in MSC NutriStem® XF Medium, the medium with the cells cultured was collected, and floating cells and cell debris were removed by using a 0.22 μm filter. For the collection of the exosome, 50-150 nm of exosome was collected directly from the cell culture medium by using a tangential flow filtration (TFF) system with a flat sheet membrane Minimate TFF Capsule with Omega 100K membrane and hollow fiber membranes HBM-TFF-EVs and HBM-TFF-MV, and then purified by using a mixed-mode column (anion exchange and molecular sieve) HiTrap Capto Core 700. Then, the exosome was purified by molecular sieve chromatography filled with Sepharose CL-6B. The collected exosome (about 2×10¹⁰ particles) was concentrated through a 100 kDa filtering membrane to 20 μL, dissolved in 0.2 mL of 0.1 N NaHCO₃ (pH 8.5), subjected to a bonding reaction with 10 mg of bifunctional chelating agent p-NCS-Bn-DOTA-GA for 5 h, then subjected to molecular sieve chromatography filled with Sepharose CL-6B with a 100 kDa filtering membrane to remove excess p-NCS-Bn-DOTA-GA, and then concentrated through a 100 kDa filtering membrane to 20 μL. The exosome modified with a bifunctional chelating group was mixed with gallium-67 or gallium-68 in a 0.1 M sodium acetate buffer (pH 5) and shaken at room temperature for 15 min, to obtain the complex of the present invention. The measured radiolabeling activity is about 97%. The complex (that is, radiolabeled gallium-67 p-NCS-Bn-DOTA-GA-exosome) of the present invention was injected intravenously into a wild-type mouse and a mouse with cerebellar atrophy. The results show that the complex of the present invention is absorbed at the cerebellum of the mouse with cerebellar atrophy by an amount about 1.5 folds higher than an amount absorbed by the wild-type mouse. The results are shown in FIG. 5. The results show that the complex gallium-67 p-NCS-Bn-DOTA-GA-exosome of the present invention can be used as a tracer for cerebellar atrophy.

### EXAMPLE 7 THERAPEUTIC EFFECT OF THE UMBILICAL CORD MESENCHYMAL STEM CELL-DERIVED EXOSOME ON THE MOUSE WITH CEREBELLAR ATROPHY

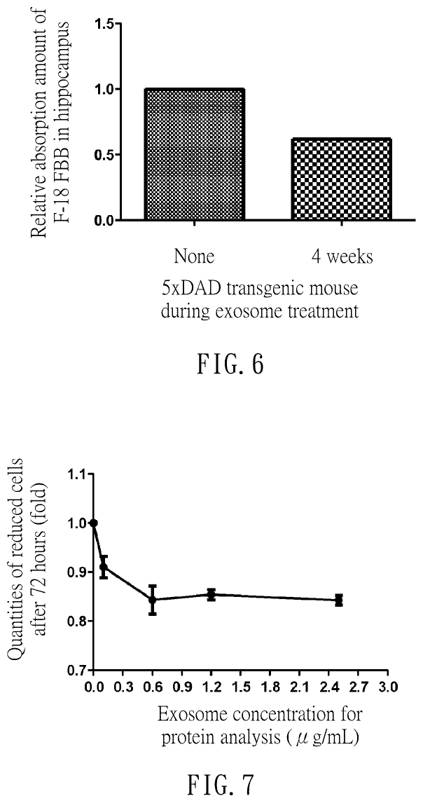

The exosome derived from the umbilical cord mesenchymal stem cells was isolated from a medium with the umbilical cord mesenchymal stem cells cultured. The umbilical cord mesenchymal stem cells were first cultured in MSC NutriStem® XF Medium, the medium with the cells cultured was collected, and floating cells and cell debris were removed by using a 0.22 μm filter. For the collection of the exosome, 50-150 nm of exosome was collected directly from the cell culture medium by using a tangential flow filtration (TFF) system with a flat sheet membrane Minimate TFF Capsule with Omega 100K membrane and hollow fiber membranes HBM-TFF-EVs and HBM-TFF-MV, and then purified by using a mixed-mode column (anion exchange and molecular sieve) HiTrap Capto Core 700. Then, the exosome was purified by molecular sieve chromatography filled with Sepharose CL-6B. The collected exosome was injected intravenously into a mouse with Alzheimer's disease by 8 μg of protein once a week for four weeks. It can be learned that amyloid deposition in the mouse with Alzheimer's disease is effectively reduced to 0.62 folds. The results are shown in FIG. 6.

### EXAMPLE 8 THERAPEUTIC EFFECT OF THE UMBILICAL CORD MESENCHYMAL STEM CELL-DERIVED EXOSOME ON THE MOUSE WITH LEUKEMIA

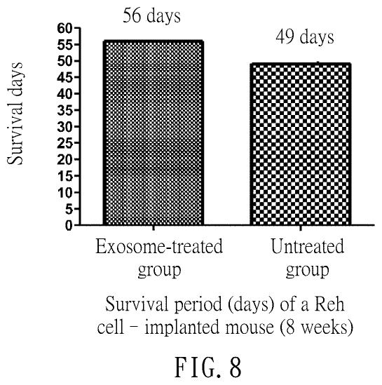

The exosome derived from the umbilical cord mesenchymal stem cells was isolated from a medium with the umbilical cord mesenchymal stem cells cultured. The umbilical cord mesenchymal stem cells were first cultured in MSC NutriStem® XF Medium, the medium with the cells cultured was collected, and floating cells and cell debris were removed by using a 0.22 μm filter. For the collection of the exosome, 50-150 nm of exosome was collected directly from the cell culture medium by using a tangential flow filtration (TFF) system with a flat sheet membrane Minimate TFF Capsule with Omega 100K membrane and hollow fiber membranes HBM-TFF-EVs and HBM-TFF-MV, and then purified by using a mixed-mode column (anion exchange and molecular sieve) HiTrap Capto Core 700. Then, the exosome was purified by molecular sieve chromatography filled with Sepharose CL-6B. The collected exosome was added into Reh leukemia cells, effectively inhibiting the growth of 70% of leukemia cells. The results are shown in FIG. 7. If this exosome is injected intravenously into a mouse with leukemia once a week for four weeks, the life span of the mouse with leukemia can be effectively prolonged by one week. This is equivalent to prolonging the life span of human beings by nine months. The results are shown in FIG. 8.

Therefore, based on the foregoing results, by the present invention, suitable patients can be first selected by using the exosome tracer, and then the exosome isolated from specific cells is applied for treatment, greatly improving the efficacy of exosome therapy. Further, the results of the foregoing examples can confirm that, by the complex and preparation method provided in the present invention, the exosome can effectively labeled with the radionuclide, so that the complex has the potential to be used as a novel diagnostic and therapeutic drug.

What is claimed is:

1. A complex, comprising:
an exosome;
a bifunctional chelating agent, coupled to the exosome, wherein the bifunctional chelating agent comprises 1,4,7,10-tetraazacyclododecane-1,4,7,10-tetraacetic acid (DOTA); and
a radionuclide, wherein the radionuclide is gallium-67 or gallium-68.

2. The complex according to claim 1, wherein the bifunctional chelating agent is p-NCS-Bn-DOTA-GA or p-NCS-Bn-DOTA.

3. The complex according to claim 1, wherein the bifunctional chelating agent is p-NCS-Bn-DOTA-GA.

4. An exosome tracer, comprising:
the complex according to claim 1; and
a pharmaceutically acceptable carrier.

5. A method for preparing the complex according to claim 1, comprising the following steps:
(a) mixing 50-150 nm of exosome with a bifunctional chelating agent for at least three hours to obtain a mixture;
(b) filtering out the excess bifunctional chelating agent to obtain an exosome binding to a bifunctional chelating group; and
(c) adding the exosome binding to the bifunctional chelating group and a radionuclide into a slightly acidic buffer solution, prior to shaking for at least 10 minutes, to obtain the complex, wherein the radionuclide is gallium-67 or gallium-68.

6. The method according to claim 5, wherein the 50-150 nm of exosome is isolated from mesenchymal stem cells.

7. The method according to claim 6, wherein a method for preparing the 50-150 nm of exosome comprises the following steps:
(a-1) collecting, by a tangential flow filtration system, a medium with the mesenchymal stem cells cultured, and collecting the 50-150 nm of exosome with a flat sheet membrane and a hollow fiber membrane; and
(a-2) purifying the 50-150 nm of exosome by column chromatography.

8. The method according to claim 7, wherein the bifunctional chelating agent is p-NCS-Bn-DOTA-GA or p-NCS-Bn-DOTA.

9. The method according to claim 8, wherein the bifunctional chelating agent is p-NCS-Bn-DOTA-GA.

10. The method according to claim 5, wherein in step (a), the 50-150 nm of exosome and the bifunctional chelating agent are mixed for at least five hours.

11. The method according to claim 5, wherein the exosome binding to the bifunctional chelating group and the radionuclide are shaken for at least 15 minutes to obtain the complex.

12. Use of the complex according to claim 1 in preparing a pharmaceutical composition for tracking severity of osteoporosis, wherein the exosome is derived from bone marrow mesenchymal stem cells.

13. Use of the complex according to claim 1 in preparing a pharmaceutical composition for tracking severity of cerebellar atrophy, wherein the exosome is derived from umbilical cord mesenchymal stem cells.

14. Use of an exosome in preparing a pharmaceutical composition for treating Alzheimer's disease or leukemia, wherein the exosome is derived from umbilical cord mesenchymal stem cells.
